(19) United States
(12) Patent Application Publication
(10) Pub. No.: US 2016/0353712 A1
(43) Pub. Date: Dec. 8, 2016

(54) Animal Urine Collection Aid

(71) Applicant: Bruce Christian Harvie, Hopewell Junction, NY (US)

(72) Inventor: Bruce Christian Harvie, Hopewell Junction, NY (US)

(21) Appl. No.: 15/164,962

(22) Filed: May 26, 2016

Related U.S. Application Data
(60) Provisional application No. 62/171,233, filed on Jun. 4, 2015.

(51) Int. Cl. A01K 23/00; A61B 10/00; E01H 1/12

(52) U.S. Cl. CPC A01K 23/005; E01H 1/12; A61B 10/007; E01H 2001/1293

(57) ABSTRACT

A plastic receptacle is designed to be used to collect urine specimens from animals for testing. It solves the problem of how to easily collect an uncontaminated urine sample from an animal, in a sanitary manner. The invention allows for collection of the urine sample from a short distance without bending over, so as not to startle the animal causing it to move or stop urinating before a sample can be collected. The device allows commonly available household items to suffice for a handle and for a specimen bottle.

### PRIORITY: CROSS-REFERENCE TO THE RELATED APPLICATION

This non-provisional utility patent application claims the benefit of and priority to U.S. Provisional Application 62/171,233 “Animal Urine Collection Aid,” filed 4 Jun. 2015.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The invention relates to a device for collecting urine specimens from domestic animals which allows commonly available household items to suffice for a handle and for a specimen bottle.

### BACKGROUND

Pets and other domestic animals sometimes require that the owner collect a sample of urine from the animal for a variety of veterinary reasons. A pet owner may wish to avoid the expense of an out-call from a veterinary professional to collect urine using special professional tools and methods, but may lack adequate means to collect a specimen himself without risk of contamination of the premises. Common instructions direct the pet owner to allow the animal to urinate on a floor or on the ground and then. attempt to collect the urine using means such as a baster or a spoon taken from the owner's own kitchen utensils.

A risk of contamination to the user exists after allowing a pet to urinate within a dwelling, and a separate risk of contamination of the sample with soil or other natural elements obtains when attempting to collect urine in contact with the ground. Furthermore, without a collection aid it is nearly impossible for an animal owner to collect fresh urine into an effective specimen container such as a bottle or a vial without the owner coming into contact with the urine.

The deficiencies of the current art include devices which expose the user to surfaces wetted with fresh urine, devices with special handles which must somehow be stored when not in use and which may only be used with the collection device, thereby taking up space when not in use for this particular activity, and devices which accept a specific vial or specimen bottle which must be procured along with the specimen collector.

The total expense of acquiring such a urine collection device therefore includes that of the collection device, its special specimen bottles or vials, and a dedicated rod or handle.

Effective use of known devices for collecting animal urine may necessitate the user coming into close proximity of an active stream of urine, or require the user to adopt an awkward body position such as stooping, deep bending, or squatting which may disturb the animal, causing it to move away from the collection device. Elderly pet owners may not have the physical flexibility to achieve or maintain an unusual body position, and some people may feel socially uncomfortable in a position they find debasing or undignified.

Some devices are intended to be positioned on the ground or on a floor and the animal is then maneuvered over the device until excretion occurs. That moment is nearly impossible to predict, and it is likely that the animal will become bored or restless when held motionless for long periods of time, taxing the owner's ability to control the animal to maintain its position over the collection device.

U.S. Pat. No. 7,214,199 “Urine Analysis Collection Kit for Veterinary Use” to Yastrebov discloses a collection device having a rectangular profile in which a bottom wall and upstanding sidewalls are claimed, and a urine collection tube or vial which must be specifically designed to mate with the device. Unlike persons involved in veterinary practice, the typical pet owner may wish to use commonly available household items as a handle to hold the device and as an expedient specimen container.

While an owner may wish to employ a handle of sufficient length to remain clear of an active urine stream, the Yastrebov device must be held directly by the user, who must enter into the immediate vicinity of the flowing urine being passed, increasing the likelihood of coming in contact with the urine.

Animals may move unpredictably when touched in their tender nether regions by objects, and the Yastrebov device as shown has a reservoir with square corners which would cause much greater discomfort to an animal unpredictably colliding with the device during a random fidget or an instinctive shake-off motion.

Because many designs fail to adequately and reliably contain excreted matter, these and other devices unfortunately require or strongly suggest that the user wears gloves during use. Many pet owners would prefer to use a device with which the risk of spills, splashes, contact, or other contamination has been mitigated to the point that they feel confident to use the device without having to wear gloves.

### BRIEF SUMMARY

In accordance with various embodiments, a convenient and sanitary device for collecting animal urine is provided. Given the deficiencies of the current state of the art, the present invention achieves a number of advantageous objectives:

In the act of collecting urine from a micturating animal, one object of the invention is to eliminate or greatly reduce the chance of the contact of the user's hands with any surface contaminated by animal urine.

Another object of the invention is to allow user to position a urine collection aid without coming into close proximity with the urine being passed, but without the user having to bend deeply, squat, or achieve other awkward or less than dignified positions.

In contrast to previous devices and as a further object of the invention, gloves are generally not required while using the invention, because the chance of direct contact by the user's hand with any surface likely to have been wetted by uresis is eliminated or greatly reduced.

Another object of the invention is to allow a user to position a urine collection aid to intercept and collect an aliquot of urine being passed without undue disturbance or discomfort caused to the animal.

Another object of the invention is that the flow path from the collection reservoir to the specimen bottle should be as short as possible, to reduce the extent of surfaces and area wetted with urine, with which the user wishes to avoid contact for reasons of basic hygiene.

Another object of the invention is that movement of the collected urine from the reservoir into the specimen bottle should be effected in as few motions as possible. The invention can transfer urine from its collection reservoir into the specimen bottle in a single act of raising the invention with a pivoting motion of the rod.

Another object of the invention is to reduce cost by providing a simple invention operable by a handle being a threaded rod taken from other implements readily available in a typical household, and using a specimen bottle which can be a commonly available beverage container or a shampoo or a lotion bottle also readily available in a typical household, thus reducing complexity and expense to the user.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows an oblique view of the invention

FIG. 2 shows a top view of the invention

FIG. 3 shows a side view of the invention engaged with a section of generic handle, and a generic bottle attached wherein the sample will eventually reside.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Since the invention is directed to the collection of liquid urine, other devices designed to collect solid waste or to collect either solid or liquid waste are outside the scope of the invention. Devices having absorbent material are also outside the scope of the invention. These devices also lack an affordance for a user-operated handle and are outside the scope of the invention. The composition of the handle and the collection or specimen bottle are also outside the scope of the invention, because one object of the invention is to allow a user to select these components from other readily available commodity products on hand.

Other devices exist outside the scope of the invention, including collection aids that include illumination, or hollow shafts or handles which transport urine.

Unlike Yastrebov, the invention is designed to accept commonly available bottles, and commonly available tool handles, rather than specific components with specific complementary coupling features which require, like Yastrebov, that the user purchase and retain all these items specifically for the purpose of animal urine collection.

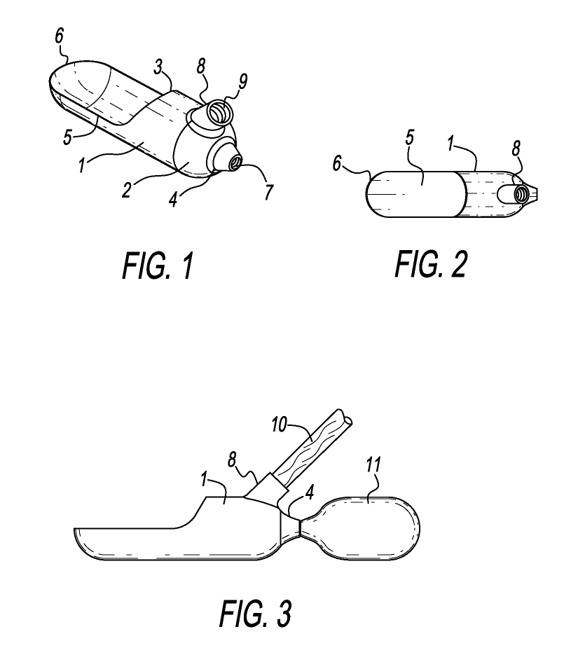

The invention is a urine collection aid to be used with pets and other domestic animals. FIG. 1 shows the form and features of the inventive collection aid [1] which includes a membrane formed into a funnel section or a fill end [2] having a larger diameter tapering to a smaller diameter, with the larger diameter also defining a rim [3]. The axis of rotational symmetry of the funnel section also defines a longitudinal direction and is a longitudinal axis. A reservoir section [5] extends from the rim in the longitudinal direction, and is closed at the far end by an end membrane [6].

Several features of the invention may be described using a broad term describing a form or feature operative within the invention, while the same feature may be described with a more exacting term when referring to the best mode shape. Thus, more broadly the invention may be described as a vessel configured to collect and contain an infalling liquid, with the vessel having a proximal fill end [2] and a distal end [6] connected by the reservoir section [5]. The span of the vessel between its proximal and distal ends defines a longitudinal axis. More narrowly and in the best mode, the fill end [2] is a funnel shape, the distal end [6] is a bulbous end membrane.

Since the animal may move of its own accord and without warning, which may cause it to collide with the collection device, it is desirable that at least the portion device in proximity with the animal's nether regions should eschew sharp corners. Although any contour suitable for collecting liquid in the reservoir may suffice as the end membrane, the bulbous shape as shown at [6] is contemplated as a best mode in that should the animal move suddenly and bump into the invention, no sharp corners are presented which may discomfort the animal. The bulbous membrane shape [6] also allows the user to interpose the device in between the animal's legs while approaching from the rear, with minimal distracting stimulus to the animal of contacted by the device in or near its more sensitive regions. However, other shapes which complete the perimeter of the collecting vessel at its distal end are included within the scope of the invention, such as a form resembling the bow of the hull of a ship, or of a spoon, or a partial cylinder as a reservoir, capped by a circular segment at its distal end.

To maintain the collection aid in an optimal position for collecting urine emitted by the animal, the collection aid is supported at a protuberance [8] adapted to accept a commonly available threaded shaft such as a broom handle or a mop handle. This protuberance emerges and extends from the proximal fill end of the vessel, and is oriented at an acute angle with respect to the longitudinal axis of the invention, so that the collecting vessel can be oriented to an optimal position while the person holding the handle can maintain a comfortable body position such as standing or nearly standing. The angle of the handle-accepting protuberance [8] orients the handle coupled to it so that control of the location of the collection aid can be effected without stooping or kneeling on the ground, and without requiring the user to remain imprudently close to an active stream of urine emitted by the animal.

In one embodiment the protuberance offers threads [9] which in its best mode are ACM ¾ inch by 5 threads per inch, which may also be specified as ¾″-5. This thread is commonly present in household mop and broom handles and also shorter handles used for paint rollers, “plumber's helper” tools, and many garden implements. When the need to collect a pet sample arises, a handle may be removed from any such tool or implement readily on hand and affixed to the invention during its use.

In contrast to Yastrebov, this protuberance [8] offered by the invention allows the user to support the collection aid with an easily secured rod or handle [10,] or a standard mop handle or broom handle generally available in a supermarket housewares section or hardware store, whereby the rod or handle allows the user to remain. more distant from the urine flow and spray and any instinctive shake-off motions which the animal may do without warning. Thus is seen that the capability of the invention to accept a handle very likely to be already present in the user's household saves expense and eliminates storage logistics required for a handle dedicated to a urine sample collector of previous designs. The bottle and a portion of the handle are shown in FIG. 3.

The fill end [2] has another threaded protuberance [4] which is hollow so that it has an orifice communicating with fill end [2] and the reservoir section [5]. In one particular set of embodiments, the fill end is a funnel section tapering from a larger diameter to a smaller diameter, with the communicating orifice emerging from a protuberance at the small end of the funnel. This orifice also has internal threads [7] adapted to accept a bottle having a threaded neck. In best modes, the axis defined by the protuberance and its orifice is longitudinally oriented; that is, substantially parallel to the longitudinal axis of the vessel and its reservoir section and, in a more particular set of embodiments these axes may be collinear and coincident.

Rather than being threaded to an obscure thread specification requiring the user to purchase specially threaded sample vials dedicated to a collector, the best mode of the invention offers a popular standard thread promulgated by the International Society of Beverage Technologists named 28/400. This thread is commonly found on beverage containers such as plastic bottles for soda drinks in 8, 12, 16, and 24 ounce sizes, but it is also readily found in numerous cosmetic bottles such as shampoo and lotion containers. Unlike previous devices, the invention having the 28/400 internal thread affords the user the opportunity to save a previously consumed container of some product or other, thereby making available for use as a specimen container an object readily available in nearly every American pet-owner's household, at no additional cost.

FIG. 2. shows a top view of the invention [1] with the previously described features so that a general concept of the overall shape of the preferred embodiment may be better understood. Proceeding from right to left in this figure, the handle-supporting threaded protuberance [8,] the reservoir section [5] in the distal collection end, with its bulbous membrane [6] are presented.

FIG. 3. shows the invention [1] with a portion of a generic handle [10] affixed to a first threaded protuberance [8,] and a generic bottle [11] threadably coupled to a second protuberance [4] of the invention by its orifice and complementary threads.

The invention is easy to use. First, a bottle on hand designated as specimen bottle is screwed into the orifice of the invention. Then a handle secured for the purpose or temporarily borrowed from another household tool on hand is screwed onto or affixed to the first protuberance of the invention.

Using the handle to hold the invention parallel with the ground, the user can then slide the invention between the legs and under animal unobtrusively, and collect a sample of urine without disturbing the animal. A simple pivoting motion from the handle will precipitate collected urine into the bottle without spreading urine over any surface likely or necessary to be handled by the user in cleaning and rinsing before disassembly. The handle can be retained during rinsing so that the user can then hold the invention and remove the handle only after urine has been fully rinsed away. The chance of a user encountering a surface wetted with urine is therefore dramatically reduced or eliminated. Furthermore, it can be understood that for improved sanitary operation, the handle-receiving protuberance should be blocked, that is, occluded from communication with the reservoir section. Were this not the case, urine in the reservoir may have a path to communicate with and contact the handle, to unsanitary effect.

It is therefore seen that the invention solves the problem of how to easily collect a sanitary uncontaminated urine sample from an animal. The invention allows for collection of the urine sample from a short distance without bending over, so as not to startle the animal causing it to stop urinating before a sample can be collected.

The invention is a very inexpensive and reusable device that connects the collection device to a readily available container for the sample to be collected into in a very sanitary way. It its best mode embodiment, its long, slender cylindrical shape it will work for the smallest of dogs and cats and also work well with larger livestock like horses etc. Some devices of related art are to large to be effectively used with smaller animals.

Comparing the invention to previous devices, when using such previous devices which only manipulate an open ended pot or dish, the problem arises that once a sample is collected the user must somehow arrange to pour It into a bottle to transport it to the veterinarian's office. This can be a sloppy, unsanitary undertaking. Other available devices are generally more expensive and although suitable for professional use, they do not lend themselves to the consumer market where the need to collect a urine sample is not something that arises very often. Thus the invention, being both very inexpensive and reusable, answers a current market need.

Other embodiments exist within the scope of the invention. For example, although the funnel and reservoir section are shown as circular in cross section, an ovoid is also contemplated within the invention. Also, while the handle-accepting protuberance is shown having internal threads, a support stud having external threads and another embodiment having no threads are also contemplated within the invention.

While certain features and aspects have been described with respect to exemplary embodiments, one skilled in the art will recognize that numerous modifications are possible. Further, while various methods and processes described herein may be described with respect to particular structural and/or functional components for ease of description, methods provided by various embodiments are not limited to any particular structural and/or functional architecture.

Hence, while various embodiments are described with or without certain features for ease of description. and to illustrate exemplary aspects of those embodiments, the various components and/or features described herein with respect to a particular embodiment can be substituted, added, and/or subtracted from among other described. embodiments, unless the context dictates otherwise. Consequently, although several exemplary embodiments are described above, it will be appreciated that the invention is intended to cover all modifications and equivalents within the scope of the following claims.

What is claimed is:

1. A urine collection aid comprising:
a vessel configured to collect and contain an infalling liquid, said vessel including a proximal fill end and a distal collection end, and said vessel extending from said fill and to said distal end along a longitudinal axis,
a first threaded protuberance extending from said proximal fill end at an angle with respect to said longitudinal axis,
a second threaded protuberance extending from said fill end of said vessel, said second threaded protuberance being hollow and terminating with an orifice which communicates with said fill end of said vessel.

2. The urine collection aid of claim 1, wherein said fill end is a funnel tapering along said longitudinal axis.

3. The urine collection aid of claim 1, wherein said distal end is a bulbous membrane.

4. The urine collection aid of claim 1, wherein said first threaded protuberance is an internally threaded protuberance.

5. The urine collection aid of claim 4, wherein said first threaded protuberance has ACME ¾″-5 internal thread.

6. The urine collection aid of claim 4, wherein said first threaded protuberance has ACME ¾″-4 internal thread.

7. The urine collection aid of claim 1, wherein said second threaded protuberance is adapted for threaded engagement with a commercially available fluid bottle.

8. The urine collection ad of claim 7, wherein the thread of said threaded protuberance is ISBT 28/400 internal thread.

9. The urine collection aid of claim 1, wherein an axis of said first threaded protuberance is coplanar with said longitudinal axis of said vessel.

10. The urine collection aid of claim 1, wherein an axis of said second threaded protuberance is substantially parallel with said longitudinal axis of said vessel.

11. The urine collection device of claim 1, further comprising a sealable translucent polymer bottle removably, threadingly engaged with said second threaded protuberance.

12. A urine collection aid comprising:
a collection vessel including a funnel section and a reservoir section, said funnel section including a rim of a first larger diameter tapering along a longitudinal axis to a smaller diameter and communicating with an orifice,
said reservoir section further comprising an arcuate membrane emerging from said rim of said larger diameter of said funnel section and extending along said longitudinal axis, and
said reservoir section terminated by a bulbous end membrane opposite the funnel section.

13. The urine collection aid of claim 12, with said orifice further including internal threads.

14. The urine collection aid of claim 13, wherein said internal thread of said orifice is ISBT 28/400 internal thread.

15. The urine collection aid of claim 12, with said orifice residing at an end of a longitudinally oriented protuberance.

16. The urine collection aid of claim 12, with said membrane further including a protuberance including an internal thread.

17. The urine collection aid of claim 16, wherein said internal thread of said protuberance is ACME ¾″-5 internal thread.

18. The urine collection aid of claim 16, wherein said internal thread of said protuberance is ACME 3/4″-4 internal thread.

19. The urine collection aid of claim 16, wherein said internally threaded protuberance is occluded from communication with said reservoir section.

20. The urine collection aid of claim 16, wherein an axis defined by said internally threaded protuberance lies at an acute angle with respect to said longitudinal axis of said reservoir section.
